(19) United States
(12) Patent Application Publication
(10) Pub. No.: US 2025/0177313 A1
(43) Pub. Date: Jun. 5, 2025

(54) FORMULATION OF INTRINSICALLY ACID-RESISTANT VEGETARIAN-BASED AND GELATIN-BASED SOFT GEL CAPSULES FOR PHARMACEUTICAL/ NUTRACEUTICAL PRODUCTS

(71) Applicant: GelEntroCeutics Inc., Winnipeg (CA)

(72) Inventors: Farzad Kobarfard, Winnipeg (CA); Marjan Esfahanizadeh, Winnipeg (CA); Arash Lotfi Seisan, Winnipeg (CA); Hooshang Lotfisisan, Winnipeg (CA)

(73) Assignee: GelEntroCeutics Inc., Winnipeg (CA)

(21) Appl. No.: 19/042,401

(22) Filed: Jan. 31, 2025

Related U.S. Application Data
(63) Continuation-in-part of application No. 17/711,602, filed on Apr. 1, 2022, now abandoned.

(30) Foreign Application Priority Data
May 18, 2021 (CA) 3119012

(51) Int. Cl. A61K 9/48

(52) U.S. Cl. CPC A61K 9/4825; A61K 9/485; A61K 9/4858; A61K 9/4866

(57) ABSTRACT

A method of manufacturing an enteric gel capsule and the enteric gel capsules produced by this method. The method of manufacturing the enteric gel capsule involves: dissolving a gelling polymer and a plasticizer into water to form an polymer mixture; mixing a bulk amount of acid insoluble polymer dispersion with a calculated amount of alkali agent to form a bulk amount of translucent acid insoluble polymer dispersion, the amount of alkali agent determined from a titration curve prepared by titrating a sample of the acid insoluble polymer; and adding the bulk amount of translucent acid insoluble polymer dispersion to the polymer mixture while mixing and heating at 70° C. until a gel mass forms.

### CROSS-REFERENCE TO RELATED APPLICATIONS

The present application is a continuation-in-part of U.S. patent application Ser. No. 17/711,602 filed on Apr. 1, 2022, which application claims priority to Canadian Patent Application No. 3,119,012 filed on May 18, 2021, both of which are incorporated herein by reference.

### FIELD

This invention is in the field of acid-resistant capsules, and in particular acid-resistant, vegetarian-based, and/or gelatin-based soft gel capsules and method of manufacturing the capsules.

### BACKGROUND

Soft gelatin capsules are used to encapsulate water-insoluble liquids dissolved in a non-polar solvent for several reasons, such as masking flavors or unpleasant smell, reducing contamination of the product and protecting the active drug against oxidation. Due to its unique functional capabilities and full compliance with the human body, gelatin is the main ingredient in soft gelatin capsules, commonly known as soft gels.

Oral pharmaceutical dosage forms with gastric resistant properties are employed to avoid degradation of the active substances by the gastric juice and also to reduce gastric irritation caused by the medicine. Enteric coated tablets have been around for decades and provided their advantages to the portions for quite a long time.

### SUMMARY

In one aspect of the present disclosure, a method of manufacturing an enteric soft gel capsule is provided. The method comprises: dissolving a gelling polymer and a plasticizer into water to form a polymer mixture; titrating a sample of an acid insoluble polymer in an aqueous dispersion with an alkali agent to obtain a titration curve; determining a first equivalence and a second equivalence from the titration curve, whereby the second equivalence is used to calculate a stoichiometric amount of alkali agent needed to neutralize a bulk amount of an acid insoluble polymer dispersion to become translucent, the bulk amount of the acid insoluble polymer to be utilized in forming an enteric gel mass for an encapsulation process; selecting the second equivalence to calculate the amount of the alkali agent to be added to the bulk amount of the acid insoluble polymer dispersion; mixing the calculated amount of the alkali agent and the bulk amount of the acid insoluble polymer dispersion to form a bulk amount of translucent acid insoluble polymer dispersion; and adding the bulk amount of the translucent acid insoluble polymer dispersion to the polymer mixture while mixing and heating at 70° C. until the enteric gel mass forms, the enteric gel mass to be utilized in the encapsulation process.

In some embodiments, the method further comprises removing bubbles from the enteric gel mass by maintaining the enteric gel mass at 50° C. for 24 hours. In some embodiments, the method further comprises removing bubbles from the enteric gel mass by placing the enteric gel mass under vacuum for up to 18 hours.

In some embodiments, the gelling polymer is selected from at least one of: a gelatin, a non-gelatin gelling agent. In some embodiments, the non-gelatin gelling agent is selected from the group consisting of: tapioca, pullulan, hydroxypropyl methylcellulose (HPMC). In some embodiments, the acid insoluble polymer is selected from the group consisting of: anionic methyl methacrylate polymer, methacrylic acid-methyl acrylate copolymer, methacrylic acid-ethyl acrylate copolymer, poly(methyl acrylate-co-methyl methacylate-co-methacrylic acid), hydroxypropyl methylcellulose phthalate (HPMCP). In some embodiments, the plasticizer is selected from at least one of: glycerol, sorbitol, triethyl citrate.

In some embodiments, an enteric soft gel capsule is provided, which enteric soft gel capsule is manufactured in accordance with any of the methods described herein. In some embodiments, the enteric soft gel capsule contains HPMC in the range of 25% wt. to 27% wt. In some embodiments, the enteric soft gel capsule contains methacrylic acid-methyl acrylate copolymer in the range of 15% wt. to 18% wt. In some embodiments, the enteric soft gel capsule contains methacrylic acid-ethyl acrylate copolymer in the range of 15% wt. to 46.5% wt.

### DESCRIPTION OF THE DRAWINGS

While the invention is claimed in the concluding portions hereof, example embodiments are provided in the accompanying detailed description which may be best understood in conjunction with the accompanying diagrams where like parts in each of the several diagrams are labeled with like numbers, and where:

FIG. 1 is a flow chart of an embodiment of the manufacturing process for the enteric soft gel capsules; and

FIG. 2 is a titration curve of methacrylic acid-ethyl acrylate copolymer.

### DETAILED DESCRIPTION

Acid resistant solid dosage forms usually have an enteric coating that prevents the disintegration and dissolution in the gastric environment. There are many enteric coated tablets in the pharmaceutical and nutraceutical market. These enteric tablets often deliver the active ingredients straight to the duodenum for intestinal absorption and they are suitable for delivering enzymatic and probiotic formulations. Manufacturing of these tablets comprises of two consecutive separate steps: First, tableting, and then coating the tablet, which is performed by application of heat and spraying the coating agents on tablets.

Soft gels are water soluble and heat sensitive and therefore they are deformed and damaged during the coating process which has to be done after the freshly produced soft capsules are made through the encapsulation process. This intensive two-step process adds time and money to the cost of each enteric capsules. Additionally, this coating produces an opaque shell which is less desired by patients.

Soft gelatin capsules are not resistant to the gastric juice and therefore acid-sensitive active pharmaceutical ingredients (API) and/or medications with gastric irritation properties cannot be formulated in a form of soft gel. Acid-resistant capsules have special formulations and ingredients that delay the release of the capsule content. The capsules often have enteric coatings that will not dissolve in the stomach juices.

The delayed release properties are the result of utilization of one of the following compounds as the coating agent: proprietary polymers, hypromellose, zein, sodium alginate, shellac, cellulose acetate trimethylate, polyvinyl acetate phthalate (PVAP), cellulose acetate succinate, cellulose acetate phthalate, hydroxypropyl methyl cellulose phthalate and an anionic methacrylate polymer such as the polymer sold under the trademark Eudragit®.

Traditionally, enteric soft gels have been prepared by coating with enteric polymers using traditional coating technology for tablets, but coating has disadvantages for soft gels such as unsuccessful adhesion for the enteric polymer onto the soft gelatin shell's inherent flexible nature. Since the coating agents are often dissolved in water and their aqueous solutions are sprayed on the soft gels while applying heat, the soft gels may be damaged and/or deformed during the process. Flaking after the drying step is another challenge for coating soft gels.

There are currently three methods for soft gel coating in pharmaceutical industries: 1—Dipping—the capsules are immersed in solution of acid-resistant polymer and then dried. 2—Pan spray—the capsules are coated in a pan coating machine in which the coating agent is sprayed at a certain temperature on the capsules. The capsules are then tumbled in the pan until they are dry. 3—Fluidized bed coating—In a fluidized bed dryer machine, the capsules are stirred and suspended in the air while coating solution and heat are applied. All three methods are challenging since they all use heat in the process.

Therefore, making gel mass with the enteric features which reside in the gel mass may provide the capability of making soft gel capsules with intrinsic acid resistant properties which resides in their shells.

As described herein, a gelatin mass containing an acid-insoluble polymer along with the other additives may make clear enteric soft gel capsules with intrinsic acid-resistance properties which resides in the capsule shell.

Acid insoluble polymers which are used for enteric coating of tablets are classified as anionic polymers containing —COOH (carboxylic acid) functional groups and thus they possess pH-dependent solubility; at a high pH, the carboxylic acid groups become ionized and make the polymers dissolve in water. At a low pH, the carboxylic acid groups are not ionized, which renders the polymer insoluble in water.

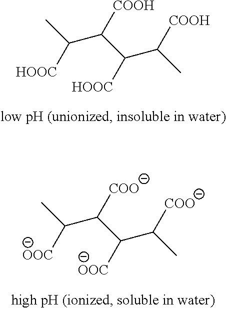

Among the above polymers, an example of an acid insoluble polymer is methacrylic acid-methyl acrylate copolymer, which is sold under the trademark “Eudragit® L”, contains an anionic copolymer based on methacrylic acid/ethyl acrylate (1:1). A further example is methacrylic acid-ethyl acrylate copolymer, which is sold under the trademark “Eudragit® L30D55”. Still a further example of such polymers is poly(methyl acrylate-co-Methyl methacylate-co-methacrylic acid).

The chemical structure of the anionic methacrylate polymer is shown below.

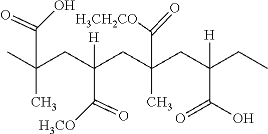

The polymer sold under the trademark Eudragit® L100 is an anionic copolymer based methacrylic acid and methyl methacrylic acid. It has acid value of 315 mg KOH/g of polymer and glass transition temperature greater than 150° C. Targeted drug release area for this polymer is jejunum and dissolves at pH above 6.

The anionic methacrylate polymer sold under the trademark Eudragit® L30D55 is the aqueous dispersion of anionic polymers with methacrylic acid as a functional group. It is a low viscosity liquid with white color with faint characteristic odor.

Eudragit® L30D55 is obtained in the form of aqueous dispersion (30%) whereas Eudragit® L100 is an anionic copolymer based on methacrylic acid and ethyl acrylate. Eudragit® L100 is a white powder with a faint characteristic odor. Both grades of Eudragit® have molecular weight 3,200,000 g/mol, acid value 315 mg KOH/g of polymer. The targeted drug release area for Eudragit® L30D55 is duodenum and Eudragit® L30D55 dissolves at pH of 5.5. Both Eudragit® L100 and Eudragit® L30 D55 are used for effective and stable coating with fast dissolution in the upper bowel, controlled release, site specific drug delivery in intestine.

Another polymer that has been utilized as an enteric coating agent is hydroxypropyl methylcellulose phthalate (HPMCP).

As described herein, combinations of one acid-insoluble polymer with a gelling agent, such as gelatin or vegetarian gelatin (tapioca) or pullulan or hydroxypropyl methylcellulose (HPMC), along with a plasticizer such as glycerin, sorbitol, and/or triethyl citrate, have been developed so that the resulting gel mass mixture could be used directly for a gel capsule encapsulation process.

The soft gel capsules which are made using these formulations have an acid-resistant shell which is not disintegrated in the acidic condition of the stomach, and are delivered to the duodenum where the capsules are disintegrated and release their contents.

These soft gel capsules have transparent or translucent appearance without needing to go through the coating process. The gel mass which is used in the process of encapsulation is intrinsically acid resistant and therefore the acid resistant soft gel is made in a single step. This one-step process is less time-consuming, less expensive and does not require a second machine for the coating process.

In one aspect of the present disclosure, acid insoluble polymers are characterized by carboxylic acid functional group and include a diverse group of polymers with variation in molecular weight, ester functional groups, and, notably, the number of carboxylic acid functional groups in their structures. As a consequence, such acid insoluble polymers exhibit distinct different neutralization capacities, leading to differences in their properties and applications. Before incorporating these polymers into the final gel mass and mixing them with gelatin and plasticizer, the Applicant has found that accurate stoichiometric neutralizing the carboxylic acid groups provides a more stable product. Otherwise, if the carboxylic acid groups are not completely neutralized, or if an excess of alkaline agent is added to the mixture, the excess base or acid in the resulting gel capsule product may have undesirable interactions between the acid groups or the excessive base and the drugs or substance contained within the gel capsule. Furthermore, the gel capsule may prematurely dissolve within the stomach, rather than passing through the stomach to the intestines.

To achieve the desired outcome, in one aspect the Applicant proposes to perform accurate titration of the acid-insoluble polymer dispersion, to precisely calculate the amount of alkaline agent required to stoichiometrically neutralize all of the carboxylic groups in the polymer dispersion. Failure to perform this titration step may result in one of two scenarios: the existence of excess alkaline agent that exceeds the acid-insoluble polymer's capacity to completely react with the alkaline agent; or the presence of remaining, non-neutralized carboxylic acid groups due to insufficient amounts of alkaline agent. Applicant has discovered that neglecting the step of performing titration, on a particular batch of acid-insoluble polymer to determine a precise stoichiometric amount of alkaline agent required to neutralize all of the carboxylic groups in the polymer, may lead to difficulties during the manufacturing process and significantly impact the stability of soft gel capsules and the API or ingredients encapsulated therein. Moreover, if the API inside the soft gel capsule is pH-sensitive, the presence of excess alkaline agent or active carboxylic acid groups in the gel capsule may further jeopardize the API's stability and result in a reduced shelf life of the final product.

While developing the novel enteric soft gel capsules, the Applicant encountered several technical challenges, including problems related to the appearance of the soft gel capsules, the encapsulation process, and the stability of the soft gel capsule itself, as well as the stability of the API contained within the capsule fill. In particular, the Applicant has identified the following problems with enteric gel capsules that are manufactured with either an excess amount or a deficient amount of alkaline agent added to the acid-insoluble polymer:

Bloom impact: High bloom gelatin provides improved soft gel capsules due to the specific requirements of these capsules. Soft gel capsules require higher strength to securely hold their liquid or semi-solid contents during handling and storage, with a reduced or eliminated risk of leaking or rupturing. The use of higher bloom gelatin provides better mechanical strength, reducing capsule rupture or leakage. Additionally, soft gel capsules ideally possess a flexible shell to accommodate various shapes and sizes of fill materials, and the higher bloom gelatin enhances the capsule's flexibility and elasticity, making it easier to handle and swallow. Moreover, soft gel capsules ideally possess a strong seal to reduce or prevent leakage of the contents. The higher bloom gelatin contributes to a more robust and reliable seal during the encapsulation process, providing for better securement of the contents within the soft gel capsule. Both incomplete neutralization and over-neutralization of the acid-insoluble polymer will impact the bloom strength of the gel mass, necessitating the management of additional challenges during the manufacturing process.

Viscosity Impact: The viscosity of the gel mass will be affected by the amount of alkaline agent added, necessitating adjustments in the manufacturing process settings, such as the film thickness of the gelatin ribbon, sealing temperature for encapsulation, and flexibility of the capsule shell.

Transparency and Appearance: The transparency and overall appearance of the soft gel capsules may be affected by the amount of alkaline agent added, requiring fine adjustments to achieve the desired visual qualities. In some embodiments, a transparent or translucent soft gel capsule is preferred from an aesthetic and a functional perspective.

Migration Pattern Change: The migration pattern of the fill content to the shell surface may be altered, potentially affecting the stability of the soft gel capsules.

Impact on pH-Sensitive APIs: In cases where pH-sensitive APIs are present in soft gel capsule, they can come into direct contact with the excess alkali materials in the shell (in the case of excessive alkali addition) or with the remaining carboxylic acid groups (in the case of insufficient alkali addition). This may initiate degradation of the API, negatively impacting the efficacy and shelf life of the soft gel capsule product.

Hence, in some embodiments, accurate and precise stoichiometric neutralization of the acid-insoluble polymer is preferred to reduce or eliminate these issues and provide for increased quality and stability of the final soft gel capsule product.

In respect of enteric coatings on formed tablets, there is a study which has indicated the deleterious effects of remaining (unneutralized) carboxylic acid functional groups in acid insoluble polymers on the stability of acid-sensitive medications like omeprazole; see A. K. Vynckier et al, “Enteric protection of naproxen in a fixed-dose combination product produced by hot-melt co-extrusion,” International Journal of Pharmaceutics, Volume 491, Issues 1-2, 1 Aug. 2015, Pages 243-249.

In one aspect of the present disclosure, the Applicant has discovered that performing the titration step on a sample of a given batch or lot of the acid-insoluble polymer, in order to accurately calculate the amount of alkaline agent required to completely neutralize the carboxylic acid groups of the acid-insoluble polymer of that particular batch or lot of the polymer, addresses these issues and provides an enteric soft gel capsule having improved stability, shelf life, and other characteristics. Preferably, the titration step should be repeated on a sample for every batch or manufacturing lot of acid-insoluble polymer, prior to using the polymer in the manufacture of soft gel capsules. This is because the precise amount of carboxyl groups may vary from batch to batch (or lot to lot) of the same acid-insoluble polymer; therefore, it is desirable to repeat the titration step for every new batch or lot of acid-insoluble polymer, to calculate the precise amount of alkaline agent required to completely neutralize all the carboxylic acid groups in a given batch of acid-insoluble polymer, while at the same time avoiding an excessive amount of alkaline agent.

The type of acid-insoluble polymer which is used in combination with gelatin or non-gelatin gelling agents (vegetarian or HPMC) and the proper composition of gelling agent and acid-insoluble polymer and plasticizer may significantly affect a quality of the shell. Encapsulation process is highly sensitive to a bloom of the combined gel and acid-resistant polymer.

Different quantities of acid-resistant polymers may be required in combination with gelatin and non-gelatin gelling agents to achieve enteric properties.

In order to increase a flexibility and an adhesion of the enteric capsules, an amount of plasticizer was adjusted, based on the acid-insoluble polymer and the gelling agent selected. Talc was not used as an anti-sticking agent so as to avoid any discoloration of a capsule surface.

Due to the lower glass transition temperature of the polymer in Eudragit® L30D, a glidant may be needed to reduce the thickness of the gel.

As described herein, a gel mass composition may be produced without requiring a coating process. The gel mass composition may comprise a gelatin or a vegetarian gelling agent (tapioca and pullulan) or hydroxypropyl methylcellulose (HPMC), in combination with an acid-insoluble polymer, for manufacturing acid-resistant (enteric) soft gel capsules.

In another aspect of the present disclosure, is the development of a non-gelatin (vegetarian) enteric gel mass is provided. This vegetarian enteric gel mass is formulated using a combination of hydroxypropylmethylcellulose (HPMC), tapioca or pullulant, all of which are non-gelatin gelling agents, and an acid-insoluble polymer, resulting in vegetarian enteric soft gel capsules.

Gelatin, tapioca, and pullulan differ significantly in terms of their viscosity, gel strength, chemical compatibility, and bloom strength:

Gelatin has a moderate to high viscosity, strong gel strength, and is chemically compatible with various ingredients.

Tapioca has low to moderate viscosity, does not form strong gels, and generally shows chemical compatibility with different substances.

Pullulan has low viscosity, moderate gel strength, and is chemically compatible with many ingredients, making it a viable alternative, particularly as a vegetarian and vegan-friendly option for encapsulation and coatings.

Therefore, the process of making an enteric gel mass for each of these gelling agents is significantly different. A single straightforward protocol used for making enteric gelatin cannot be directly extended to the non-gelatin versions of the enteric soft gel capsules. Instead, specific coarse and fine adjustments and modifications must be applied to tailor the process for each individual gelling agent. This ensures that the desired enteric properties are achieved effectively for each material in the soft gel capsule manufacturing process.

For instance, HPMC differs significantly from gelatin; it is a synthetic, inert, and water-soluble polymer derived from cellulose, possessing distinct viscosity, molecular weight, and functional group characteristics compared to gelatin.

During our experimental investigations, the Applicant conducted various compatibility studies between Hydroxypropylmethylcellulose (HPMC) and HPMCP, the chosen acid-insoluble polymer. These experiments revealed that HPMC demonstrated a higher level of compatibility with HPMCP as compared to the Methacrylate polymers. This compatibility between HPMC and HPMCP facilitates achieving a cohesive and stable gel mass for vegetarian enteric softgels.

While either HPMCP and Methacrylate polymer may be utilized in combination with HPMC to create the vegetarian enteric gel mass, the Applicant observed that these different options for acid-insoluble polymers involve distinct treatment methods during the formulation process. As a result, in some embodiments it is preferable to develop a universal protocol that may be applied to both types of polymers, ensuring consistent and reliable results.

To achieve this, the Applicant focused on determining the acid value of HPMCP through a titration process. By precisely determining the acid value of HPMCP, information about its neutralization capacity and its compatibility with HPMC is obtained. This knowledge provided the ability to achieve optimal performance and ensure uniformity in the gel mass preparation when using either HPMCP or Methacrylate polymer in conjunction with HPMC for vegetarian enteric soft gel capsules.

Overall, our comprehensive approach allowed us to overcome the challenges associated with using different acid-insoluble polymers and HPMC in the development of vegetarian enteric gel masses. It resulted in a reliable and standardized protocol that enhances the quality, stability, and effectiveness of the final vegetarian enteric soft gel capsule products.

The following are a few examples of percent compositions to achieve enteric properties of soft gel capsules; such examples are provided for illustrative purposes only, and are not intended to be limiting.

### Example 1

| | % wt. |
|---|---|
| Gelatin | 35% |
| Water | 35% |
| Glycerol | 15% |
| HPMCP | 15% |
| NaOH | 3.375 g |

### Example 2

| | % wt. |
|---|---|
| Gelatin | 32.5% |
| Water | 32.5% |
| Glycerol | 17.5% |
| HPMCP | 17.5% |
| NaOH | 3.95 g |

### Example 3

| | % wt. |
|---|---|
| Gelatin | 35% |
| Water | 35% |
| Glycerol | 15% |
| HPMCP | 15% |
| NH₄OH sol. 25% | 5.7 ml |

### Example 4

| | % wt. |
|---|---|
| Gelatin | 32.5% |
| Water | 32.5% |
| Glycerol | 17.5% |
| HPMCP | 17.5% |
| NH₄OH sol. 25% | 6.65 ml |

### Example 5

| | % wt. |
|---|---|
| HPMC | 25% |
| Water | 45% |
| Glycerol | 15% |
| Eudragit ® L100 | 15% |
| NaOH | 3.375 g |

### Example 6

| | % wt. |
|---|---|
| HPMC | 25% |
| Water | 45% |
| Glycerol | 15% |
| Eudragitt ® L100 | 15% |
| NH₄OH sol. 25% | 5.7 ml |

### Example 7

| | % wt. |
|---|---|
| HPMC | 25% |
| Water | 30% |
| Glycerol | 15% |
| Eudragitt ® L30D55 | 30% |
| NaOH | 2.25 g |

### Example 8

| | % wt. |
|---|---|
| HPMC | 25% |
| Water | 35% |
| Glycerol | 15% |
| Eudragitt ® L30D55 | 25% |
| NH₄OH sol. 25% | 3.8 ml |

### Example 9

| | % wt. |
|---|---|
| HPMC | 27% |
| Water | 31.3% |
| Glycerol | 17.1% |
| Eudragitt ® L30D55 | 19.8% |
| NH₄OH sol. 25% | 4.8% |

### Example 10

| | % wt. |
|---|---|
| Gelatin | 27% |
| Water | 30.4% |
| Glycerol | 18% |
| Triethylcitrate | 1.8% |
| Eudragitt ® L100 | 18% |
| NH₄OH sol. 25% | 4.8% |

### Example 11

| | % wt. |
|---|---|
| Gelatin | 25% |
| Water | 3.9% |
| Glycerol | 19% |
| Triethylcitrate | 1.8% |
| Eudragitt ® L30D5 | 45.5% |
| NH₄OH sol. 25% | 4.8% |

### Example 12

| | % wt. |
|---|---|
| Gelatin | 33.5% |
| Water | 10.2% |
| Glycerol | 17.8% |
| Eudragitt ® L30D5 | 36% |
| NaOH | 2.5% |

### Gel Making Process

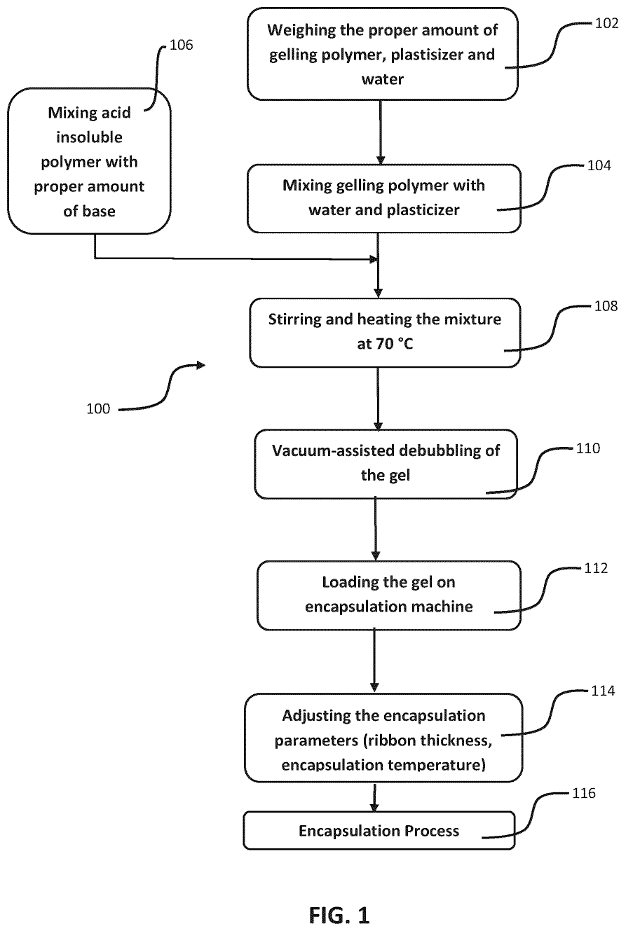

Turning to FIG. 1, the gel capsule manufacturing process 100 starts by weighing a specific amount of a gelling agent (otherwise referred to as a gelling polymer), plasticizer, and water at step 102, with the gelling agent, plasticizer and water mixed together at step 104 to form the polymer mixture. An acid insoluble polymer may be dissolved or suspended in water and mixed with a predetermined amount of an alkali agent at step 106, with the predetermined amount of alkali agent determined by creating a titration curve of the acid insoluble polymer and selecting a second equivalence from the titration curve. Thus, at step 106, the film forming polymer is formed. The film forming polymer may be mixed with the polymer mixture, while stirring and heating the mixture at 70° C. at step 108 until a gel mass is formed; in some cases, the heating and stirring at step 108 may occur for approximately 2 hours. In some cases, a gel mass may be maintained at 60° C. overnight.

The temperature was then brought to 50° C. and the gel mass may be kept at this temperature for 24 hours to remove any bubbles. Alternatively, such as at step 110, the gel mass may be kept under vacuum for 18 hours to remove any bubbles. The gel mass so obtained may be directly used in encapsulation process. The gel mass may be loaded on an encapsulation machine at step 112. One or more encapsulation parameters may be adjusted at step 114, such as ribbon thickness, encapsulation temperature, etc. Then, the encapsulation process may be performed at step 116.

In some embodiments, an optimum formulation for the manufacturing process for an amount of the alkaline reagent (e.g. NaOH or NH₄OH solution) which may be added to the acid insoluble polymer, such as Eudragit® L30D55 (methacrylic acid-Ethyl acrylate copolymer). In order to calculate the required amount of alkaline agent, the methacrylic acid-Ethyl acrylate copolymer (or other suitable acid insoluble polymer) may be titrated as described herein.

As an illustrative example, not intended to be limiting, the pH titration may be performed on the methacrylic acid-Ethyl acrylate copolymer dispersed in water. For example, a volume of 10 ml of the polymer suspension at 0.1% wt may be titrated using, for example, 0.1% wt NaOH solution. The titration may be conducted under stirring, at room temperature, and a pH was measured as a function of the NaOH volume added.

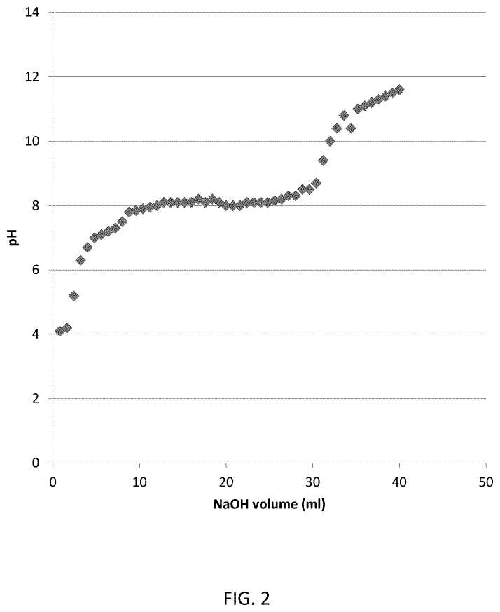

Referring to one example of a titration curve, as shown in FIG. 2, the titration curve of the methacrylic acid-ethyl acrylate copolymer obtained from direct pH-titration revealed two equivalence points. The presence of two equivalence points is in agreement with a polyacid character of the methacrylic acid-ethyl acrylate copolymer and it was observed irrespective of the polymer titration amount. The polymer may not be soluble until the pH of first equivalence since the solutions remained turbid.

When a first equivalence is reached, the polymer is not totally solubilized, which means that a dissociation carboxylic groups amount is not sufficient to ensure a total solubilization of the polymer and the medium remains turbid. During a second equivalence, the solution becomes translucent indicating that the amount of dissociated carboxylic functions is enough to ensure the solubilization of the polymer.

The second equivalence may be used to estimate an amount of carboxylic functions on the polymer, and may therefore be used to calculate the predetermined amount of alkaline agent to be added to the acid insoluble polymer when forming the film forming polymer mixture. This amount of alkaline agent to be added varies with changing the source of methacrylic acid ethyl acrylate copolymer (sold under the trademark Eudragit®), or other type of acid insoluble polymer. For methacrylic acid-ethyl acrylate copolymer, an estimated amount of carboxylic acid functional groups on the polymer, in the titration of one sample of one batch, was calculated as 6 mmol/g.

A total amount of alkaline reagent to be used for each formulation may be calculated based on the titration of water insoluble polymer (methacrylic acid ethyl acrylate copolymer).

### In Vitro Dissolution Studies

Using an United States Pharmacopeia (USP) apparatus 2 at 50 rpm, in 900 ml of medium at 37° C. with a wire sinker was used. Two hours of exposure in 0.1 N HCl (pH=1.2) was followed by testing in 0.05 M phosphate buffer of pH 6.8. The capsules that adhere to USP dissolution were accepted as enteric capsules. Capsules may not release more than 10% of the fill during two hours in 37° C. simulated gastric fluid. Capsules may be fully dissolved by 45 min in simulated intestinal fluid.

The foregoing is considered as illustrative only of the principles of the invention. Further, since numerous changes and modifications will readily occur to those skilled in the art, it is not desired to limit the invention to the exact construction and operation shown and described, and accordingly, all such suitable changes or modifications in structure or operation which may be resorted to are intended to fall within the scope of the claimed invention.

1. A method of manufacturing an enteric soft gel capsule, comprising:
dissolving a gelling polymer and a plasticizer into water to form a polymer mixture;
titrating a sample of an acid insoluble polymer in an aqueous dispersion with an alkali agent to obtain a titration curve;
determining a first equivalence and a second equivalence from the titration curve, whereby the second equivalence is used to calculate a stoichiometric amount of alkali agent needed to neutralize a bulk amount of an acid insoluble polymer dispersion to become translucent, the bulk amount of the acid insoluble polymer to be utilized in forming an enteric gel mass for an encapsulation process,
selecting the second equivalence to calculate the stoichiometric amount of the alkali agent to be added to the bulk amount of the acid insoluble polymer dispersion;
mixing the calculated stoichiometric amount of the alkali agent and the bulk amount of the acid insoluble polymer dispersion to form a bulk amount of translucent acid insoluble polymer dispersion; and
adding the bulk amount of the translucent acid insoluble polymer dispersion to the polymer mixture while mixing and heating at 70° C. until the enteric gel mass forms, the enteric gel mass to be utilized in the encapsulation process.

2. The method of claim 1, further comprising:
removing bubbles from the enteric gel mass by maintaining the enteric gel mass at 50° C. for 24 hours.

3. The method of claim 1, further comprising:
removing bubbles from the enteric gel mass by placing the enteric gel mass under vacuum for up to 18 hours.

4. The method of claim 1 wherein the gelling polymer is selected from at least one of: a gelatin, a non-gelatin gelling agent.

5. The method of claim 4, wherein the non-gelatin gelling agent is selected from the group consisting of: tapioca, pullulan, hydroxypropyl methylcellulose (HPMC).

6. The method of claim 1 wherein the acid insoluble polymer is selected from the group consisting of: anionic methyl methacrylate polymer, methacrylic acid-methyl acrylate copolymer, methacrylic acid-ethyl acrylate copolymer, poly(methyl acrylate-co-methyl methacrylate-co-methacrylic acid), hydroxypropyl methylcellulose phthalate (HPMCP).

7. The method of claim 1 wherein the plasticizer is selected from at least one of: glycerol, sorbitol, triethyl citrate.

8. An enteric soft gel capsule manufactured according to the method of claim 5, wherein the enteric soft gel capsule contains HPMC in the range of 25% wt. to 27% wt.

9. An enteric soft gel capsule manufactured according to the method of claim 6, wherein the enteric soft gel capsule contains methacrylic acid-methyl acrylate copolymer in the range of 15% wt. to 18% wt.

10. An enteric soft gel capsule manufactured according to the method of claim 8, wherein the enteric soft gel capsule contains methacrylic acid-methyl acrylate copolymer in the range of 15% wt. to 18% wt.

11. An enteric soft gel capsule manufactured according to the method of claim 6, wherein the enteric soft gel capsule contains methacrylic acid-ethyl acrylate copolymer in the range of 15% wt. to 46.5% wt.

12. An enteric soft gel capsule manufactured according to the method of claim 8, wherein the enteric soft gel capsule contains methacrylic acid-ethyl acrylate copolymer in the range of 15% wt. to 46.5% wt.
